(19) United States
(12) Patent Application Publication
(10) Pub. No.: US 2025/0281404 A1
(43) Pub. Date: Sep. 11, 2025

(54) COMPOSITION FOR USE IN PRESSURISED DISPENSING CONTAINER

(71) Applicant: Recipharm Laboratories, Inc., Morrisville, NC (US)

(72) Inventors: Lei Mao, Morrisville, NC (US); Nischal Pant, Morrisville, NC (US)

(21) Appl. No.: 18/599,091

(22) Filed: Mar. 7, 2024

(51) Int. Cl. A61K 9/12; A61K 9/00; A61K 31/137; A61K 47/10; A61M 15/00; A61P 11/06; A61P 11/08

(52) U.S. Cl. CPC A61K 9/124; A61K 9/008; A61K 31/137; A61K 47/10; A61M 15/0065; A61P 11/06; A61P 11/08

(57) ABSTRACT

The present invention provides a composition for use in a pressurised dispensing container, the composition comprising:
from 3 to 10 wt. % of a monohydric alcohol, based on the total weight of the composition;
at least 85 wt. % HFA152a and/or HFO1234ze, based on the total weight of the composition; and
albuterol sulfate and/or levalbuterol tartrate.

The present invention relates to a composition for use in a pressurised dispensing container, and in particular to a composition for use in a pharmaceutical metered dose aerosol inhaler device.

Conventional pressurised dispensing containers, such as those used in pharmaceutical metered dose aerosol inhaler devices, typically comprise propellants such as chlorofluorocarbons (CFCs) and hydrofluoroalkanes (HFAs). Transitioning from CFCs to HFAs has contributed to successful recovery of the ozone layer. However, concerns still remain over the use of HFAs, such as HFA134a and HFA227ea, due to their global warming potential (GWP) and long atmospheric life (AL).

There is therefore a desire in the field and pressure from regulatory organisations to develop compositions for use in pressurised dispensing containers comprising alternative propellants, such as those having lower GWP and shorter AL. In particular, there is a desire and pressure to provide devices that can sustainably supply such life-saving medicines to patients, such as in pharmaceutical metered dose aerosol inhaler devices. However, it is not known whether formulations similar to those which contain the currently preferred propellants would still be suitable for use with new propellants.

The present invention seeks to tackle at least some of the problems associated with the prior art or at least to provide a commercially acceptable alternative solution thereto.

The present invention provides a composition for use in a pressurised dispensing container, a pressurised dispensing container, a pharmaceutical dispensing device, a composition for use in the treatment of bronchospasm and/or asthma and a method of treating bronchospasm and/or asthma according to the claims appended hereto.

Specifically, in a first aspect, the present invention provides a composition for use in a pressurised dispensing container, the composition comprising:
from 3 to 10 wt. % of a monohydric alcohol, based on the total weight of the composition;
at least 85 wt. % HFA152a and/or HFO1234ze, based on the total weight of the composition; and
albuterol sulfate and/or levalbuterol tartrate.

The inventors have surprisingly found that it is possible to use compositions according to the invention in a pressurised dispensing container to provide performance comparable to conventional devices, even after storage, but containing propellants having lower GWP and shorter AL. Compositions comprising the balance of components as described herein may be capable of being used in pressurised dispensing containers to provide a good balance of overall performance properties such that they may be comparable to products currently on the market, which contain propellants having higher GWP and longer AL.

It will be appreciated that the actuator with which the pressurised dispensing container is used can be modified to adjust certain performance characteristics, but, notwithstanding this, it has been found that the compositions of the invention may facilitate providing a device capable of having such comparable performance characteristics. Without wishing to be bound by theory, it is thought that this may be due to the particular monohydric alcohol, such as ethanol, content of the composition in combination with the newly investigated propellants.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

HFA152a is also known as 1,1-Difluoroethane. HFO1234ze is also known as 1,3,3,3-Tetrafluoropropene. Each are propellants having lower GWP and shorter AL than those typically used in current compositions for use in pressurised dispensing containers, and in particular pharmaceutical metered dose aerosol inhaler devices. For example, a typical propellant that is currently used is HFA-134a, which is also known as 1,2,2,2-tetrafluoroethane.

The composition is typically an inhalable composition.

Monohydric alcohols are typical excipients for the actives used in such compositions, the active being the albuterol sulfate and/or levalbuterol tartrate in this case. In an alternative embodiment, the composition comprises an alternative active other than albuterol sulfate and/or levalbuterol tartrate, such as an alternative pharmaceutically acceptable derivative or salt of albuterol or levalbuterol.

As will be appreciated, conventional albuterol is a racemic mixture of(S)-albuterol and (R)-albuterol (levalbuterol). Without wishing to be bound by theory, it is thought that levalbuterol may be the therapeutically active component of albuterol whereas(S)-albuterol is considered inert with some unwanted effects. However, it is common in the field to use albuterol.

The invention will now be described in relation to the following non-limiting drawings in which:

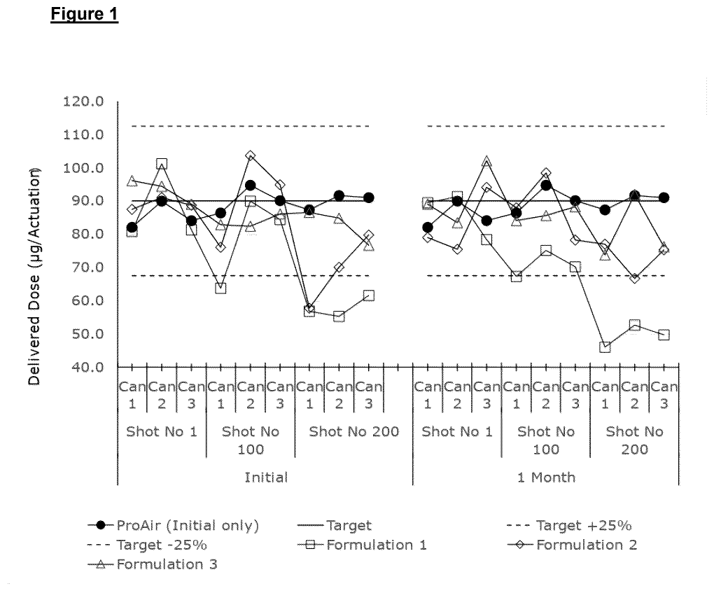

FIG. 1 shows the delivered dose results from Example 1A, comparing the prepared Formulations with the marketed product ProAir.

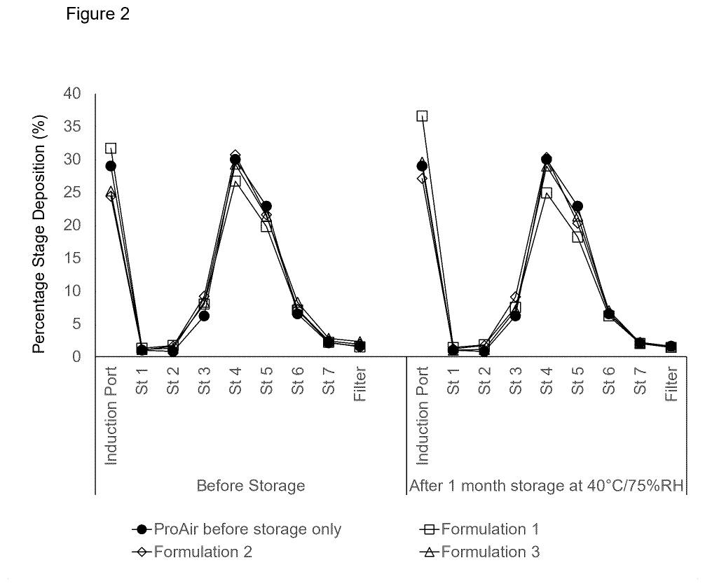

FIG. 2 shows the aerodynamic particle size distribution by next generation impactor results from Example 1B, comparing the prepared Formulations with the marketed product ProAir.

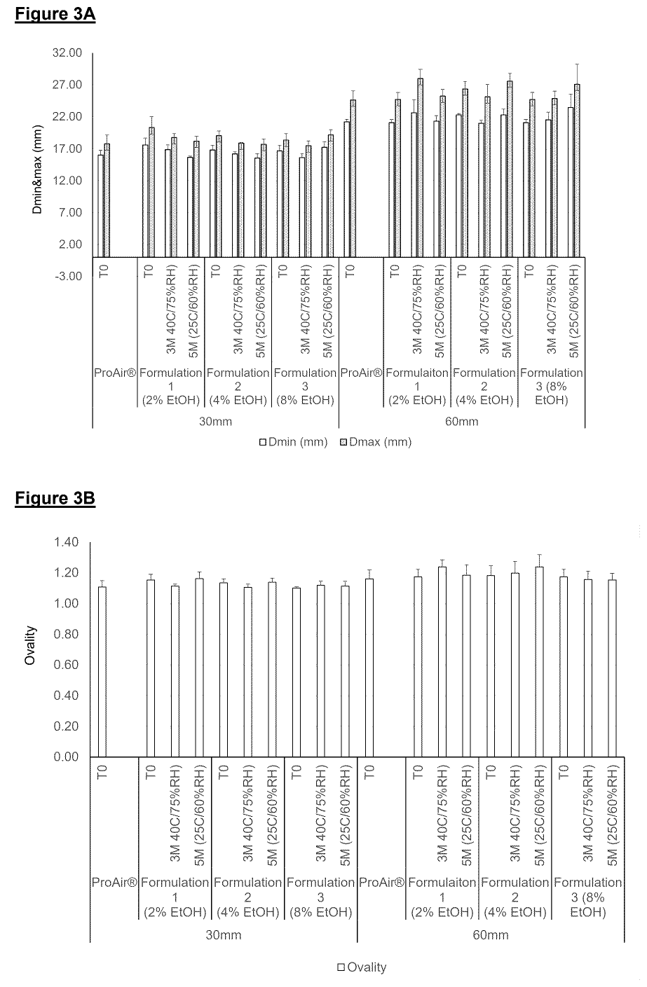

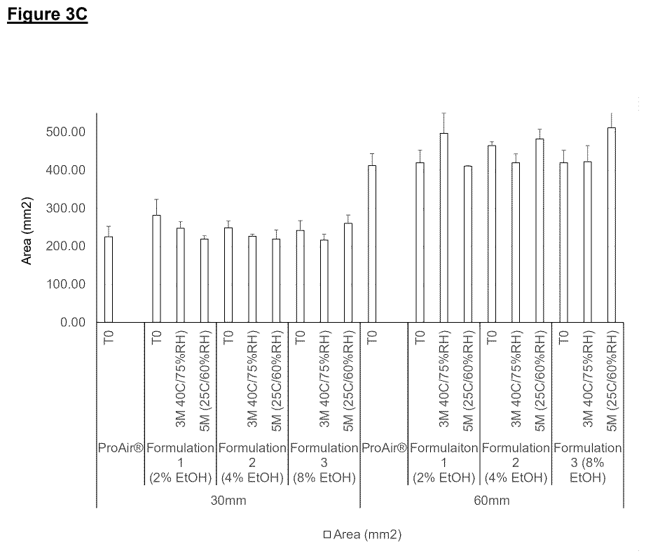

FIGS. 3A-3C show the spray pattern results from Example 1C, comparing the prepared Formulations with the marketed product ProAir. In particular, FIG. 3A shows the Dₘᵢₙ and Dₘₐₓ, FIG. 3B shows the ovality and FIG. 3C shows the area, at 30 mm and 60 mm.

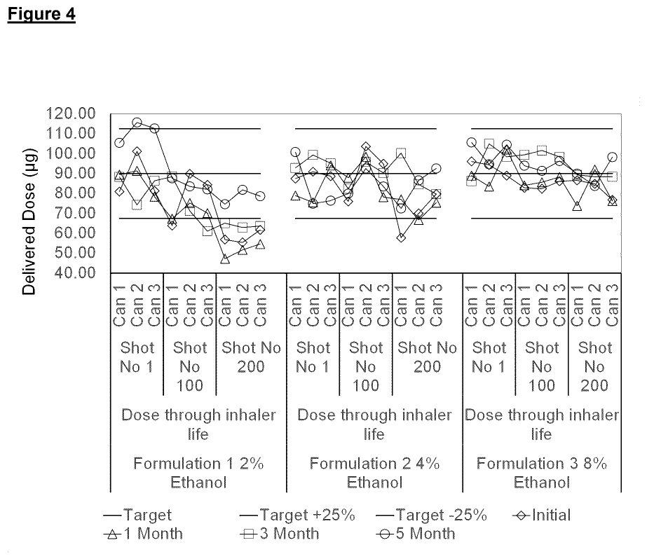

FIG. 4 shows the delivered dose results from Example 2A, comparing the prepared Formulations after longer storage times than in Example 1A.

Preferably, the monohydric alcohol comprises a C1-C3 monohydric alcohol, i.e. a monohydric alcohol comprising from one to three carbon atoms, more preferably the monohydric alcohol comprises ethanol, and even more preferably the monohydric alcohol is ethanol. The monohydric alcohol preferably consists of ethanol.

Preferably, the hydrofluorocarbon propellant of the composition comprises, and more preferably is, HFA152a. In other words, the composition preferably comprises at least 85 wt. % HFA152a, based on the total weight of the composition. That is, out of HFA152a and/or HFO1234ze, HFA152a is preferred. In other words, preferably, the composition comprises at least 85 wt. % HFA152a and/or HFO1234ze, based on the total weight of the composition, and the HFA152a and/or HFO1234ze is only HFA152a. In other words, the hydrofluorocarbon propellant of the composition preferably consists of HFA152a.

Preferably, the albuterol sulfate and/or levalbuterol tartrate is micronized, i.e. preferably the albuterol sulfate and/or levalbuterol tartrate comprises, preferably is, micronized albuterol sulfate and/or micronized levalbuterol tartrate.

Preferably, the active in the composition comprises, and more preferably is, albuterol sulfate. In other words, the composition preferably comprises albuterol sulfate. That is, out of albuterol sulfate and/or levalbuterol tartrate, albuterol sulfate is preferred. In other words, the active in the composition preferably consists of albuterol sulfate.

Accordingly, a preferred embodiment of the invention is a composition for use in a pressurised dispensing container, the composition comprising:
from 3 to 10 wt. % ethanol, based on the total weight of the composition;
at least 85 wt. % HFA152a, based on the total weight of the composition; and
albuterol sulfate.

The composition preferably comprises from 3 to 9 wt. % of the monohydric alcohol, preferably from 4 to 8 wt. % of the monohydric alcohol, based on the total weight of the composition. It has been found that, surprisingly, a monohydric alcohol content of from 4 to 8 wt. %, in particular, in combination with the newly investigated propellants, can facilitate providing a good balance of overall performance properties. In some preferred embodiments, for example if a consistent delivered dose of the active across the life of the product is the target performance characteristic for a particular use, the composition preferably comprises from 5 to 10 wt. % of the monohydric alcohol, more preferably from 6 to 9 wt. %, even more preferably from 6 to 8 wt. %, still more preferably from 7 to 8 wt. %, based on the total weight of the composition. In other preferred embodiments, for example if a consistent mean fine particle fraction and mean mass media aerodynamic diameter of the particles of the spray after storage is the target performance characteristic for a particular use, the composition preferably comprises from 3 to 7 wt. % of the monohydric alcohol, more preferably from 3 to 6 wt. %, even more preferably from 4 to 6 wt. %, still more preferably from 4 to 5 wt. %, based on the total weight of the composition.

The composition preferably comprises from 0.1 to 1 wt. % of the albuterol sulfate and/or levalbuterol tartrate, preferably from 0.2 to 0.6 wt. % of the albuterol sulfate and/or levalbuterol tartrate, based on the total weight of the composition. Such an amount of the active is preferred to deliver the desired dosage in use.

Preferably, the ratio of the albuterol sulfate and/or levalbuterol tartrate to the monohydric alcohol is from 1 to 30 wt. %, preferably from 2 to 20 wt. %, more preferably from 5 to 10 wt. %. Such a weight ratio of these components of the composition may assist in achieving the advantageous performance characteristics described herein, such as achieving the desired delivered dose, for example.

Preferably, the composition comprises at least 89 wt. % of the HFA152a and/or HFO1234ze, preferably at least 91 wt. % of the HFA152a and/or HFO1234ze, based on the total weight of the composition. Preferably, the composition comprises from 85 to 97 wt. % of the HFA152a and/or HFO1234ze, based on the total weight of the composition, more preferably from 89 to 96 wt. %, even more preferably from 91 to 96 wt. %, even more preferably from 91 to 9.59 wt. %, still more preferably from 91 to 95.5 wt. %.

The composition preferably comprises:
from 4 to 8 wt. % of the monohydric alcohol;
from 91 to 95.9 wt. % of the HFA152a and/or HFO1234ze; and
from 0.1 to 1 wt. % of the albuterol sulfate and/or levalbuterol sulfate, based on the total weight of the composition.

More preferably, the composition comprises:
from 4 to 8 wt. % ethanol;
from 91 to 95.9 wt. % HFA152a; and
from 0.1 to 1 wt. % albuterol sulfate, based on the total weight of the composition.

The composition may preferably further comprise a surfactant, preferably one or more of oleic acid, polyethylene glycol, diethylene glycol monoethyl ether, polyoxyethylene sorbitan monolaurate, polyoxyethylene sorbitan monooleate, propoxylated polyethylene glycol and polyoxyethylene. Most preferably, when the composition further comprises a surfactant, the composition further comprises oleic acid. That is, the surfactant preferably is oleic acid.

Preferably, if present, the composition comprises less than 2 wt. % of the surfactant, more preferably less than 1 wt. % of the surfactant, and even more preferably less than 0.5 wt. % of the surfactant, based on the total weight of the composition. For example, preferably, if present, the composition comprises from 0.01 to 2 wt. % of the surfactant, more preferably from 0.05 wt. % to 1 wt. % of the surfactant, and even more preferably from 0.1 to 0.5 wt. % of the surfactant, based on the total weight of the composition.

The composition preferably consists essentially of, preferably consists of:
from 3 to 10 wt. % of the monohydric alcohol, preferably from 4 to 8 wt. % of the monohydric alcohol, based on the total weight of the composition;
from 0.1 to 1 wt. % of the albuterol sulfate and/or levalbuterol tartrate, preferably from 0.2 to 0.6 wt. % of the albuterol sulfate and/or levalbuterol tartrate, based on the total weight of the composition; and
the balance being HFA152a and/or HFO1234ze, together with any unavoidable impurities.

In another preferred embodiment, the composition consists essentially of, preferably consists of:
from 3 to 10 wt. % of the monohydric alcohol, preferably from 4 to 8 wt. % of the monohydric alcohol, based on the total weight of the composition;
from 0.1 to 1 wt. % of the albuterol sulfate and/or levalbuterol tartrate, preferably from 0.2 to 0.6 wt. % of the albuterol sulfate and/or levalbuterol tartrate, based on the total weight of the composition;
optionally from 0.01 to 2 wt. % of a surfactant, based on the total weight of the composition; and
the balance being HFA152a and/or HFO1234ze, together with any unavoidable impurities.

More preferably, the composition consists essentially of, preferably consists of:
from 3 to 10 wt. % ethanol, preferably from 4 to 8 wt. % ethanol, based on the total weight of the composition;
from 0.1 to 1 wt. % albuterol sulfate, preferably from 0.2 to 0.6 wt. % albuterol sulfate, based on the total weight of the composition; and
the balance being HFA152a, together with any unavoidable impurities.

In another preferred embodiment, the composition consists essentially of, preferably consists of:
from 3 to 10 wt. % ethanol, preferably from 4 to 8 wt. % ethanol, based on the total weight of the composition;
from 0.1 to 1 wt. % albuterol sulfate, preferably from 0.2 to 0.6 wt. % albuterol sulfate, based on the total weight of the composition;
optionally from 0.01 to 2 wt. % oleic acid, based on the total weight of the composition; and
the balance being HFA152a, together with any unavoidable impurities.

As will be appreciated, the term “consists essentially of” or “consisting essentially of” as used herein may encompass that further components other than those listed may be present, provided that they do not materially affect the essential characteristics of the composition.

Preferably, the composition is capable of providing comparable delivered dose performance compared to corresponding formulations comprising HFA-134a as the propellant, preferably throughout the life of the pressurised dispensing container, preferably wherein the delivered dose remains consistent throughout the life of the pressurised dispensing container, such as within 20%, preferably within 10%, of the initial delivered dose measurements.

In a further aspect, the present invention provides a pressurised dispensing container containing the composition of any preceding claim.

Preferably, the pressurised dispensing container is pressurised to a pressure of from 300 kPa to 15 MPa, such as from 400 kPa to 5 MPa, even more preferably from 500 kPa to 3.5 MPa.

Preferably, the pressurised dispensing container is fluorinated ethylene propylene coated, plasma coated and/or polyester coated, more preferably plasma coated. That is, the internal surfaces of the pressurised dispensing container are preferably at least partially fluorinated ethylene propylene coated, plasma coated and/or polyester coated, more preferably plasma coated. Suitable coatings are known in the field.

In a further aspect, the present invention provides a pharmaceutical dispensing device comprising the pressurised dispensing container of the above aspect, preferably wherein the pharmaceutical dispensing device is a pharmaceutical metered dose aerosol inhaler device. The device of the invention is not particularly limited, provided that it is suitable for use with the composition of the invention. The device, such as the actuator of the device, may be modified further to optimise certain performance characteristics, such as the spray pattern.

Preferably, the composition described herein is for use in the treatment of bronchospasm and/or asthma. Alternatively, in a further aspect the present invention provides the use of the composition described herein for the manufacture of a medicament for the treatment of bronchospasm and/or asthma.

In a further aspect, the present invention provides a method of treating bronchospasm and/or asthma comprising administering to a subject an effective amount of the composition described herein. Preferably, the subject is a human.

Preferably, the administering to a subject an effective amount of the composition comprises using the pharmaceutical dispensing device of the above aspect.

The invention will now be described in relation to the following non-limiting examples.

### EXAMPLE 1

### Formulations and Testing Procedures

Formulations were prepared having compositions as defined in Table 1. Each formulation was prepared by filling micronized albuterol sulfate suspensions into plasma coated cans crimped with 25 μL valves. All canisters were quarantined valve down at room temperature for no less than 14 days and then on stability at 40° C./75% relative humidity (RH) for one month.

TABLE 1

| | | Per can | | |
|---|---|---|---|---|
| | Ethanol | Albuterol | | |
| Formulation | (wt. %) | sulfate (mg) | Ethanol (g) | HFA152a (g) |
| 1 | 2 | 30 | 0.14 | 6.8 |
| 2 | 4 | 30 | 0.28 | 6.7 |
| 3 | 8 | 30 | 0.56 | 6.4 |

In the following Examples, the properties in use of Formulations 1-3 were compared with the marketed product ProAir (hereinafter PA), a known albuterol sulfate suspension metered dose inhaler (MDI) comprising the propellant HFA-134a. The formulations were tested before and after one month's storage.

In particular, coated cannisters containing the same valve as each other, but the different formulations 1-3, were tested for delivered dose (DD), aerodynamic particle size distribution (APSD) by Next Generation Impactor (NGI) and spray pattern (SP), and compared against the conventional PA product. In other words, the PA product was used as a standard by which to compare. The same actuator as in the PA product was used for all tests with the PA and the formulations 1-3. United States Pharmacopeia (USP) equipment was used to the collect DD and NGI samples at 30 L/min, which were then analysed by high performance liquid chromatography (HPLC). SprayVIEW (Proveris Scientific Corporation, USA) was used to measure SP.

### 1A—Delivered Dose

Three canisters for each Formulation were tested for DD at the beginning, middle and end of inhaler life (BOL, i.e. shot no. 1, MOL, i.e. shot no. 100, and EOL, i.e. shot no. 200), before and after the one month's storage. These results were compared with initial results for PA (i.e. only without one month's storage). Formulation 3 having 8 wt. % ethanol delivered more consistent doses with means of 86.5 μg and 86.1 μg and an overall range of 73.8 μg to 102.1 μg (within +/−5% of the mean values) before and after storage, respectively, compared to 88.6 μg from the PA. Formulations with 2 wt. % (Formulation 1) and 4 wt. % (Formulation 2) ethanol exhibited lower EOL doses, but the DD for Formulation 2 was still acceptable. The DD for Formulation 2 was also more consistent across the life of the canisters compared to that for Formulation 1, and exhibited a lower reduction in MOL doses, particularly after one month's storage.

FIG. 1 illustrates the delivered doses from Formulations 1, 2 and 3 containing 2 wt. %, 4 wt. % and 8 wt. % ethanol, respectively, before (left chart) and after (right chart) one month's storage valve down at 40° C./75% RH in comparison with that from PA (results before storage only). Little change was observed for the delivered dose through inhaler life before and after storage with Formulations 2 and 3 (containing 4 wt. % and 8 wt. % ethanol, respectively) showing more consistent doses.

### 1B—Aerodynamic Particle Size Distribution (APSD) by Next Generation Impactor (NGI)

Three canisters for each Formulation were tested at both BOL and EOL for APSD by NGI. FIG. 2 and Table 2 present percentage stage deposition normalized against the DD. Formulations 2 and 3 (having 4 and 8 wt. % ethanol, respectively) showed the most comparable performance to PA. It is thought that the slightly higher induction port deposition in the lower ethanol formulations could be caused by higher velocities due to higher vapour pressures. Little change in the APSD was observed after storage.

TABLE 2

| | | NGI Stage; Deposition (%, mean of n = 3) | | | | | | | | |
|---|---|---|---|---|---|---|---|---|---|---|
| Time | | Induction | | | | | | | | |
| point | Formulation | port | 1 | 2 | 3 | 4 | 5 | 6 | 7 | Filter |
| Initial | PA | 29.0 | 1.0 | 0.8 | 6.2 | 30.0 | 22.9 | 6.5 | 2.1 | 1.6 |
| Initial | 2 | 24.4 | 1.0 | 1.6 | 9.2 | 30.7 | 21.6 | 7.2 | 2.4 | 1.9 |
| | 1 | 31.7 | 1.3 | 1.7 | 8.0 | 26.7 | 19.8 | 7.1 | 2.2 | 1.5 |
| | 3 | 25.2 | 1.1 | 1.3 | 8.3 | 29.3 | 21.4 | 8.3 | 2.8 | 2.3 |
| One | 2 | 27.1 | 1.2 | 1.7 | 9.1 | 30.3 | 20.3 | 6.6 | 2.2 | 1.6 |
| month | 1 | 36.6 | 1.4 | 1.8 | 7.5 | 24.9 | 18.2 | 6.2 | 2.0 | 1.4 |
| | 3 | 28.2 | 1.0 | 1.1 | 7.1 | 29.0 | 21.4 | 7.0 | 2.1 | 1.6 |

FIG. 2 illustrates percentage deposition (%) of the dose delivered from Formulations 1, 2 and 3 containing 2 wt. %, 4 wt. % and 8 wt. % ethanol, respectively, before (left chart) and after (right chart) one month's storage valve down at 40° C./75% RH in comparison with that from PA (results before storage only). Little change was observed for the percentage deposition of all three Formulations.

### 1C-Spray Pattern (SP)

FIG. 3 shows the Dₘᵢₙ and Dₘₐₓ (FIG. 3A), ovality (FIG. 3B), and area (FIG. 3C) at 30 mm and 60 mm, after different storage conditions.

At T0, i.e. before storage, Formulation 3 having 8 wt. % ethanol had a good match to the PA in the Dₘᵢₙ, Dₘₐₓ, ovality and area with a percentage difference of 4.1%, 3.3%, 0.7% and 7.4%, respectively, at 30 mm, and 0.7%, 0.3%, 1.1% and 1.9%, respectively, at 60 mm. This meets a typical in vitro bioequivalence (IVBE) of less than 10% for a generic MDI. Formulation 2 is also comparable, but Formulation 1 has a higher percentage difference.

FIGS. 3A, 3B and 3C also show data after three months' storage at 40° C./75% RH and five months' storage at 25° C./60% RH (see Example 2) in comparison with those from PA (before storage only). Little change was observed before and after storage for all three Formulations and the results are similar to those measured from PA.

### EXAMPLE 2

The formulations of Example 1 were then stored for longer periods of time and some of the same tests were conducted. In particular, Formulations 1-3 were also each stored for three months at 40° C./75% RH and five months at 25° C./60% RH. The following data were collected.

### 2A—Delivered Dose

The same testing as in Example 1A, now with the samples after longer storage, was conducted and the results are shown in FIG. 4.

Similarly to with the shorter storage periods, Formulation 3 containing 8 wt. % ethanol showed consistent doses throughout the life of the inhaler, with means of 86.5 μg at initial (i.e. after no time in storage), 86.1 μg and 95.0 μg after 1 and 3 month's storage at 40° C./75% RH, respectively, and 95.4 μg after 5 month's storage at 25° C./60% RH. This is compared with 88.6 μg from the PA.

Formulations with 2 wt. % (Formulation 1) and 4 wt. % (Formulation 2) ethanol exhibited lower EOL doses, but the DD for Formulation 2 was still acceptable. The DD for Formulation 2 was also more consistent across the life of the canisters compared to that for Formulation 1, and exhibited a lower reduction in MOL doses, particularly after one and three month's storage at 40° C./75% RH.

### 2B—Aerodynamic Particle Size Distribution (APSD) by Next Generation Impactor (NGI),

The same testing as in Example 1B, now with the samples after longer storage, was conducted and the results are shown in Table 3. In particular, Table 3 shows the mean fine particle dose (FPD), mean fine particle fraction (FPF), mean mass media aerodynamic diameter (MMAD) and mean geometric standard deviation (GSD). Fine particles are those less than 5 μm in diameter. Standard deviation is given in parentheses.

Table 3 shows that the FPF, MMAD and GSD were similar before and after storage for each formulation, but closer to PA for formulations 2 and 3. However, slightly greater reduction in FPF and increase MMAD after storage was seen for Formulation 3.

TABLE 3

| | Storage | Time | FPD (μg) | | FPF (%) | | MMAD (μm) | | GSD Mean | |
|---|---|---|---|---|---|---|---|---|---|---|
| Formulation | Condition | Point | Mean (SD) | | Mean (SD) | | Mean (SD) | | (SD) | |
| PA | N/A | T0 | 59.3 | (2.1) | 66.9 | (3.2) | 2.4 | (0) | 1.5 | (0) |
| 2 | N/A | T0 | 58.5 | (6.3) | 69.3 | (5.5) | 2.5 | (0.1) | 1.6 | (0.1) |
| | 40° C./75% RH | 1 M | 56.5 | (9.5) | 66.3 | (6.5) | 2.5 | (0.1) | 1.6 | (0.1) |
| | | 3 M | 63.9 | (9.7) | 71.7 | (6.7) | 2.6 | (0.1) | 1.5 | (0.1) |
| | 25° C./60% RH | 5 M | 54.8 | (6.3) | 64.3 | (1.8) | 2.6 | (0.1) | 1.7 | (0.3) |
| 1 | N/A | T0 | 42.7 | (7.7) | 62.2 | (8.2) | 2.5 | (0.1) | 1.7 | (0.1) |
| | 40° C./75% RH | 1 M | 42.3 | (10.7) | 57.2 | (5.6) | 2.5 | (0.1) | 1.7 | (0.1) |
| | | 3 M | 49.5 | (12.1) | 58 | (6.2) | 2.7 | (0.1) | 1.5 | (0.2) |
| | 25° C./60% RH | 5 M | 44.2 | (6.3) | 56.6 | (3.8) | 2.6 | (0.1) | 1.7 | (0.1) |
| 3 | N/A | T0 | 58.5 | (3.7) | 69.2 | (5.2) | 2.4 | (0) | 1.6 | (0) |
| | 40° C./75% RH | 1 M | 55.0 | (6.1) | 65.5 | (3.6) | 2.4 | (0) | 1.6 | (0) |
| | | 3 M | 67.6 | (18.5) | 65.7 | (5.3) | 2.7 | (0.1) | 1.5 | (0.1) |
| | 25° C./60% RH | 5 M | 45.8 | (3.7) | 58.7 | (3.6) | 2.5 | (0.1) | 1.6 | (0.1) |

In summary, formulation 3 containing 8 wt. % ethanol gave a consistent DD through inhaler life but a slightly reduced FPF and a larger MMAD after storage, whereas formulation 2 containing 4 wt. % ethanol showed fewer changes in the FPF and MMAD but slightly lower end of life doses. Considering both DD and APSD performance, the preferred formulations may therefore comprise from 4 to 8 wt. % of a monohydric alcohol, such as ethanol. That is, surprisingly, it has been found that the monohydric alcohol (i.e. ethanol) content of the formulations is critical in controlling the delivery performance, and that a content of from 3 to 10 wt. %, such as from 4 to 8 wt. %, may provide a good balance of overall performance properties in order to be capable of being comparable to current formulations containing propellants having higher GWP and longer AL.

The formulations of the invention can therefore be used to deliver consistent doses and provide comparable performance to PA in terms of the percentage deposition and APSD by NGI, as well as spray pattern, even after storage.

The foregoing detailed description has been provided by way of explanation and illustration, and is not intended to limit the scope of the appended claims. Many variations in the presently preferred embodiments illustrated herein will be apparent to one of ordinary skill in the art and remain within the scope of the appended claims and their equivalents.

The invention claimed is:

1. A composition for use in a pressurised dispensing container, the composition comprising:
from 3 to 10 wt. % of a monohydric alcohol, based on the total weight of the composition;
at least 85 wt. % HFA152a and/or HFO1234ze, based on the total weight of the composition; and
albuterol sulfate and/or levalbuterol tartrate.

2. The composition of claim 1, wherein the monohydric alcohol comprises a C1-C3 monohydric alcohol, preferably ethanol.

3. The composition of claim 1, wherein the hydrofluorocarbon propellant comprises HFA152a.

4. The composition of claim 1, wherein the composition comprises albuterol sulfate.

5. The composition of claim 1, comprising from 3 to 9 wt. % of the monohydric alcohol, preferably from 4 to 8 wt. % of the monohydric alcohol, based on the total weight of the composition.

6. The composition of claim 1, wherein the composition comprises from 0.1 to 1 wt. % of the albuterol sulfate and/or levalbuterol tartrate, preferably from 0.2 to 0.6 wt. % of the albuterol sulfate and/or levalbuterol tartrate, based on the total weight of the composition.

7. The composition of claim 1, wherein the ratio of the albuterol sulfate and/or levalbuterol tartrate to the monohydric alcohol is from 1 to 30 wt. %, preferably from 2 to 20 wt. %, more preferably from 5 to 10 wt. %.

8. The composition of claim 1, comprising at least 89 wt. % of the HFA152a and/or HFO1234ze, preferably at least 91 wt. % of the HFA152a and/or HFO1234ze, based on the total weight of the composition.

9. The composition of claim 1, comprising:
from 4 to 8 wt. % of the monohydric alcohol;
from 91 to 95.9 wt. % of the HFA152a and/or HFO1234ze; and
from 0.1 to 1 wt. % of the albuterol sulfate and/or levalbuterol sulfate, based on the total weight of the composition.

10. The composition of claim 1, further comprising a surfactant, preferably one or more of oleic acid, polyethylene glycol, diethylene glycol monoethyl ether, polyoxyethylene sorbitan monolaurate, polyoxyethylene sorbitan monooleate, propoxylated polyethylene glycol and polyoxyethylene.

11. The composition of claim 1, wherein the composition consists essentially of, preferably consists of:
from 3 to 10 wt. % of the monohydric alcohol, preferably from 4 to 8 wt. % of the monohydric alcohol, based on the total weight of the composition;
from 0.1 to 1 wt. % of the albuterol sulfate and/or levalbuterol tartrate, preferably from 0.2 to 0.6 wt. % of the albuterol sulfate and/or levalbuterol tartrate, based on the total weight of the composition;
optionally from 0.01 to 2 wt. % of a surfactant, based on the total weight of the composition; and
the balance being HFA152a and/or HFO1234ze, together with any unavoidable impurities.

12. A pressurised dispensing container containing the composition of claim 1.

13. A pharmaceutical dispensing device comprising the pressurised dispensing container of claim 12, preferably wherein the pharmaceutical dispensing device is a pharmaceutical metered dose aerosol inhaler device.

14. The composition of claim 1, for use in the treatment of bronchospasm and/or asthma.

15. A method of treating bronchospasm and/or asthma comprising administering to a subject an effective amount of the composition of claim 1.

16. The method of claim 15, wherein the administering to a subject an effective amount of the composition comprises using a pharmaceutical dispensing device comprising a pressurised dispensing container containing the composition, preferably wherein the pharmaceutical dispensing device is a pharmaceutical metered dose aerosol inhaler device.
